(19) United States
(12) Patent Application Publication
(10) Pub. No.: US 2020/0281512 A1
(43) Pub. Date: Sep. 10, 2020

(54) IMAGING APPARATUS AND METHOD OF IMAGING BLOOD VESSELS IN A TARGET REGION OF TISSUE

(71) Applicant: Smith & Nephew PLC, Watford, Hertfordshire (GB)

(72) Inventors: Scott Grubb, Cambridge (GB); Peter Laitenberger, Cambridge (GB)

(21) Appl. No.: 16/491,901

(22) PCT Filed: Mar. 9, 2018
(86) PCT No.: PCT/EP2018/055951

§ 371 Date: Sep. 6, 2019

(30) Foreign Application Priority Data
Mar. 9, 2017 (GB) 1703769.8

(51) Int. Cl. A61B 5/1455; A61B 5/00

(52) U.S. Cl. CPC A61B 5/14552; A61B 5/489

(57) ABSTRACT

In some embodiments, imaging apparatus for imaging blood vessels within a target region of tissue includes a housing having an aperture which, in use, is placed against a target region of tissue such that the target region of tissue occludes the aperture, a light source arranged to illuminate at least a portion of a target region of tissue occluding the aperture and to provide illuminating light having predetermined first and second spectral ranges which are different, an imaging device arranged to receive illuminating light reflected by the target region of tissue occluding the aperture, wherein the imaging device is configured to generate image output at the first and second spectral ranges, and a controlling element arranged to selectively control the imaging device and/or the light source to sequentially capture at least one image at the first spectral range and at least one image at the second spectral range.

This disclosure relates to imaging apparatus and a method of imaging blood vessels within a target region of tissue.

Wound healing is natural process performed by the human body in response to injury. The amount of time taken for a wound to heal is dependent on many different factors which include the human body's ability to heal itself and any treatments that are applied to the wound to accelerate wound healing. Understanding the healing status of a wound and being able to monitor the healing process helps to inform decisions on further treatment of the wound and can also assist in the development of future wound therapies.

One factor that is known to be associated with wound healing is the amount of blood that is supplied to blood vessels, such as capillaries, within tissue at or near a wound. The process of supplying blood to blood vessels within tissue is known as blood perfusion. Oxygen and nutrients carried by blood within wounded tissue are essential for wound healing and so the amount of blood oxygenated blood within tissue is known to correlate well with wound healing. Conventional techniques for determining the presence of blood within skin tissue require bulky specialist equipment that is typically expensive.

It is an aim of the present disclosure to at least partly mitigate the above-mentioned problems.

It is an aim of certain embodiments of the present disclosure to image blood vessels within a target region of tissue so that blood vessels within the tissue are clearly identifiable.

It is an aim of certain embodiments of the present disclosure to image blood vessels within a target region of tissue so that blood vessels carrying oxygenated blood within the tissue can be distinguished from blood vessels carrying deoxygenated blood.

It is an aim of certain embodiments of the present disclosure to image blood vessels within a target region of tissue and to distinguish blood vessels from other features within, on or comprising part of the skin tissue.

It is an aim of certain embodiments of the present disclosure to provide a compact apparatus for imaging blood vessels within a target region of tissue.

According to some embodiments, there is provided an imaging apparatus for imaging blood vessels within a target region of tissue, comprising: a housing having an aperture which, in use, is placed against a target region of tissue such that the target region of tissue occludes the aperture; a light source arranged to illuminate at least a portion of a target region of tissue occluding the aperture, the light source is configured to provide illuminating light having a predetermined first spectral range and to provide illuminating light having a predetermined second spectral range which is different from the first spectral range; an imaging device arranged to receive illuminating light reflected by the target region of tissue occluding the aperture, wherein the imaging device is configured to generate an image output at the first spectral range and an image output at the second spectral range; and a controller or controlling element arranged to selectively control the imaging device and/or the light source to sequentially capture at least one image generated by the imaging device at the first spectral range and at least one image generated by the imaging device at the second spectral range.

The imaging apparatus may be configured such that the first spectral range and the second spectral range do not overlap.

The imaging device may be configured to detect light across a continuous spectral range which encompasses the first spectral range and the second spectral range.

The controlling element may be configured to control the light source to provide illuminating light at only the first spectral range during capture of the at least one image at the first spectral range and to provide illuminating light at only the second spectral range during capture of the at least one image at the second spectral range.

The first spectral range may correspond to a spectral range associated with visible light. Visible light may be regarded as light having a wavelength between 380 nm and 770 nm.

The first spectral range may correspond to a spectral range associated with visible red light. Visible red light may be regarded as light having a wavelength of between 600 nm and 750 nm.

The second spectral range may correspond to a spectral range encompassing a wavelength absorbed by blood.

The second spectral range may correspond to a spectral range associated with infrared light.

The second spectral range may correspond to light having a wavelength between 850 nm and 1000 nm.

The imaging device may comprise a charge coupled device and/or a complementary metal-oxide semiconductor.

The light source may comprise at least a first light emitter configured to emit light having the first spectral range and a second light emitter configured to emit light having the second spectral range.

The controlling element may be configured to selectively activate the first light emitter during capture of the image at the first spectral range. The controlling element may be configured to selectively activate the second light emitter during capture of the image at the second spectral range.

The light source may be configured to emit light having a spectral range which encompasses the first spectral range and the second spectral range.

The imaging device may comprise at least a first filter arranged to transmit light within the first spectral range and a second filter arrange to transmit light within the second spectral range, wherein the controlling element is configured to selectively apply the first filter during capture of the image at the first spectral range and to apply the second filter during capture of the image at the second spectral range.

The housing may be a rigid housing and the imaging device is secured to the housing such that, when the aperture is placed against the target region of tissue, the imaging device is spaced from the target region of tissue by a predetermined distance.

The imaging device may comprise a lens having a predefined focal length and the predetermined distance is such that the lens is spaced from the target region of tissue by a distance that is equal to the focal length.

The housing may be opaque to light having a wavelength which is within the first spectral range and may be opaque to light having a wavelength which is within the second spectral range.

The housing may define a light path extending from the light source to the aperture and from the aperture to the imaging device and the housing is arranged to shield the light path from ambient light.

The light source and/or the imaging device may be disposed within the housing.

The apparatus may be a hand-held device comprising the light source, the imaging device, the housing and the integrated screen.

According to some embodiments, there is provided an imaging apparatus for imaging blood vessels within a target region of tissue, comprising: a housing having an aperture which, in use, is placed against a target region of tissue such that the target region of tissue occludes the aperture; a light source arranged to illuminate at least a portion of a target region of tissue occluding the aperture, the light source is configured to provide illuminating light having a predetermined first spectral range and to provide illuminating light having a predetermined second spectral range which is different from the first spectral range; an imaging device arranged to receive illuminating light reflected by the target region of tissue occluding the aperture, wherein the imaging device is configured to generate an image output at the first spectral range and an image output at the second spectral range; and a controller or controlling element arranged to selectively control the imaging device and/or the light source to sequentially capture at least one image generated by the imaging device at the first spectral range and at least one image generated by the imaging device at the second spectral range, the controlling element further arranged to combine the at least one image captured at the first spectral range and the at least one image captured at the second spectral range to produce a composite image in which blood vessels within the target region of skin tissue can be distinguished.

According to some embodiments, there is provided a method of imaging blood vessels within a target region of tissue using an imaging apparatus comprising a housing having an aperture, comprising the steps: holding the housing against a target region of tissue such that the target region of tissue occludes the aperture; illuminating the target region of tissue occluding the aperture using light having at least a predetermined first spectral range; capturing at least one image of the target region of tissue at the first spectral range; illuminating the target region of tissue using light having at least a predetermined second spectral range which is different from the first spectral range; and capturing at least one image of the target region of tissue at the second spectral range.

The light having at least the predetermined first spectral range may be light having only the first spectral range.

The light having at least the predetermined second spectral range may be light having only the second spectral range.

The step of capturing at least one image of the target region of tissue at the first spectral range comprises the step of filtering the light having at least the first spectral range such that only light having the first spectral range is transmitted for image capture.

The step of capturing at least one image of the target region of tissue at the second spectral range may comprise the step of filtering the light having at least the second spectral range such that only light having the second spectral range is transmitted for image capture.

The first spectral range and the second spectral range may not overlap.

The first spectral range may correspond to a spectral range associated with visible light. The first spectral range may correspond to a spectral range associated with visible red light.

The second spectral range may correspond to a spectral range encompassing a wavelength absorbed by blood. The second spectral range may correspond to a spectral range associated with infrared light. The second spectral range may correspond to a light having a wavelength between 850 nm and 1000 nm.

The method may further comprise the step of automatically combining the at least one image captured at the first spectral range and the at least one image captured at the second spectral range to produce a composite image in which blood vessels within the target region of skin tissue can be distinguished.

According to some embodiments, there is provided a method of imaging blood vessels within a target region of tissue using an imaging apparatus comprising a housing having an aperture, comprising the steps: illuminating a target region of tissue occluding the aperture of the housing held against the target region of tissue using light having at least a predetermined first spectral range; capturing at least one image of the target region of tissue at the first spectral range; illuminating the target region of tissue using light having at least a predetermined second spectral range which is different from the first spectral range; and capturing at least one image of the target region of tissue at the second spectral range.

The light having at least the predetermined first spectral range may be light having only the first spectral range.

The light having at least the predetermined second spectral range may be light having only the second spectral range.

The step of capturing at least one image of the target region of tissue at the first spectral range comprises the step of filtering the light having at least the first spectral range such that only light having the first spectral range is transmitted for image capture.

The step of capturing at least one image of the target region of tissue at the second spectral range may comprise the step of filtering the light having at least the second spectral range such that only light having the second spectral range is transmitted for image capture.

The first spectral range and the second spectral range may not overlap.

The first spectral range may correspond to a spectral range associated with visible light. The first spectral range may correspond to a spectral range associated with visible red light.

The second spectral range may correspond to a spectral range encompassing a wavelength absorbed by blood. The second spectral range may correspond to a spectral range associated with infrared light. The second spectral range may correspond to a light having a wavelength between 850 nm and 1000 nm.

The method may further comprise the step of automatically combining the at least one image captured at the first spectral range and the at least one image captured at the second spectral range to produce a composite image in which blood vessels within the target region of skin tissue can be distinguished.

According to some embodiments, there is provided a method of imaging blood vessels within a target region of tissue using an imaging apparatus comprising a housing having an aperture, comprising the steps: illuminating a target region of tissue occluding the aperture of the housing held against the target region of tissue using light having at least a predetermined first spectral range; capturing at least one image of the target region of tissue at the first spectral range; illuminating the target region of tissue using light having at least a predetermined second spectral range which is different from the first spectral range; capturing at least one image of the target region of tissue at the second spectral range; and automatically combining the at least one image captured at the first spectral range and the at least one image captured at the second spectral range to produce a composite image in which blood vessels within the target region of skin tissue can be distinguished.

Certain embodiments of the present disclosure allow for images to be obtained of a target region of tissue in which blood vessels within the tissue are identifiable.

Certain embodiments of the present disclosure allow for images to be obtained of a target region of tissue in which blood vessels carrying oxygenated blood within the tissue are distinguishable from blood vessels carrying deoxygenated blood within the tissue.

Certain embodiments of the present disclosure allow for a portable handheld device to be provided which comprises readily available components, typically referred to as off-the-shelf components, for imaging blood vessels within skin tissue. Such a device is convenient to use and relatively inexpensive.

Embodiments of the present disclosure will now be described, by way of example only, with reference to the accompanying drawings in which:

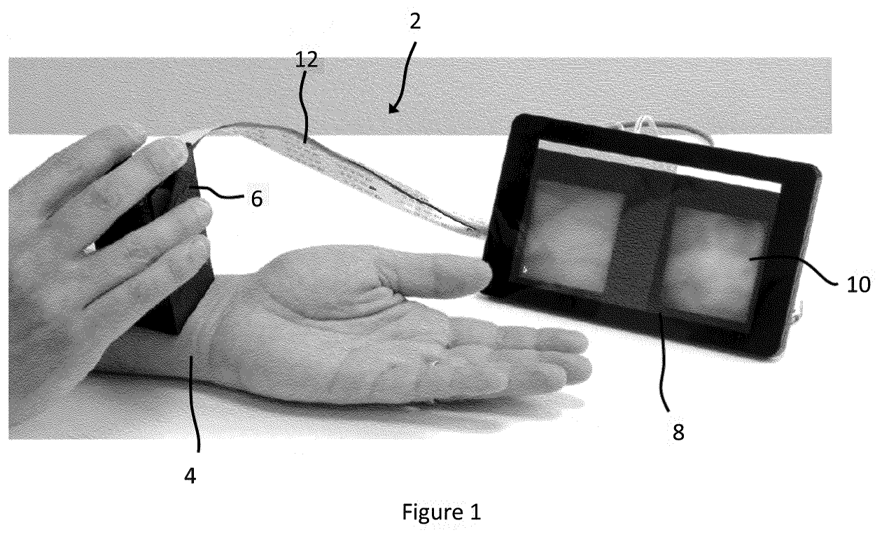

FIG. 1 shows imaging apparatus for imaging a target region of tissue;

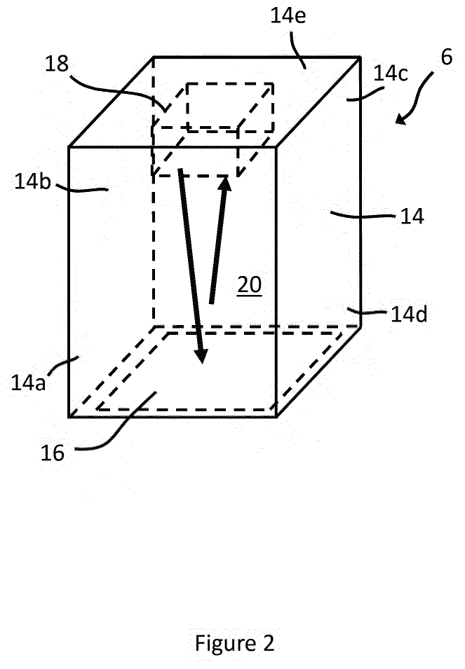

FIG. 2 is a schematic representation of part of the imaging apparatus shown in FIG. 1 comprising an imaging unit;

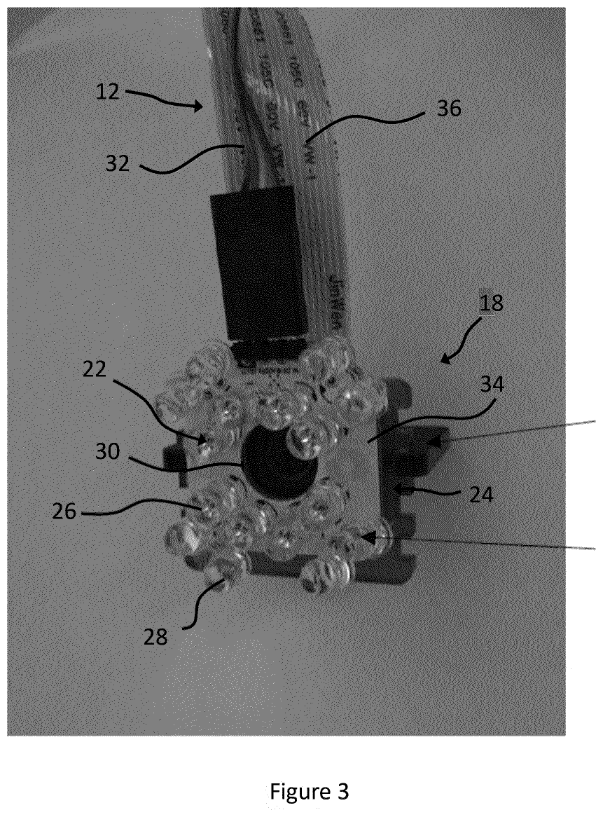

FIG. 3 shows a portion of the imaging apparatus shown in FIGS. 1 and 2 comprising a sensor module;

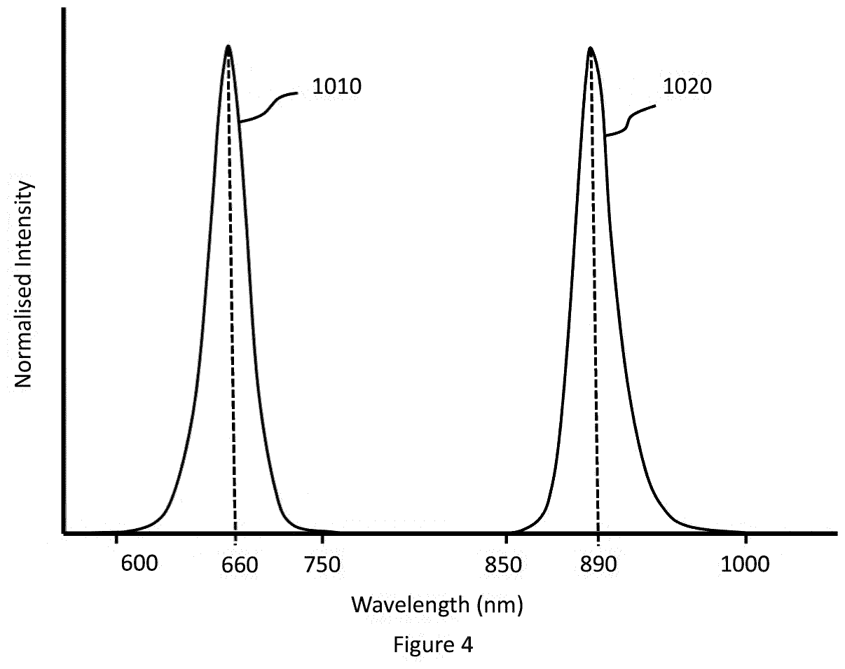

FIG. 4 shows spectral distributions for the sensor module shown in FIG. 3;

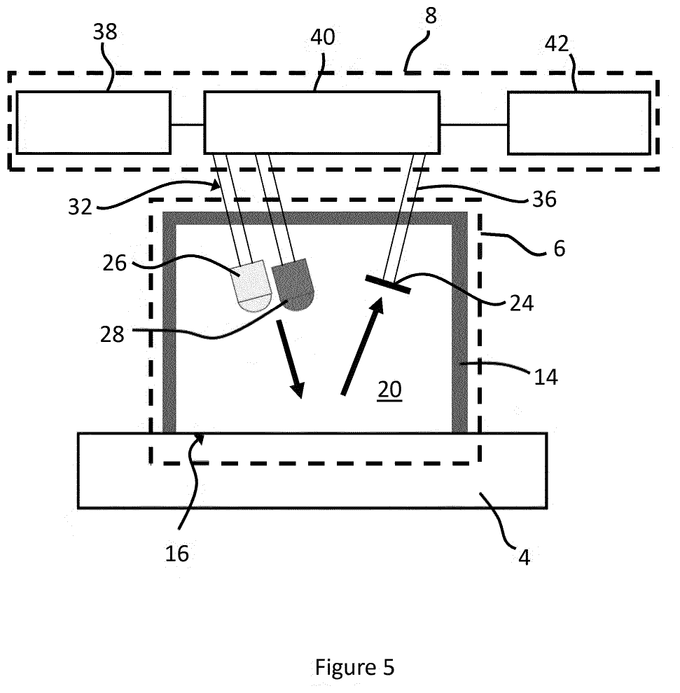

FIG. 5 is a schematic representation of key components of the apparatus shown in FIG. 1;

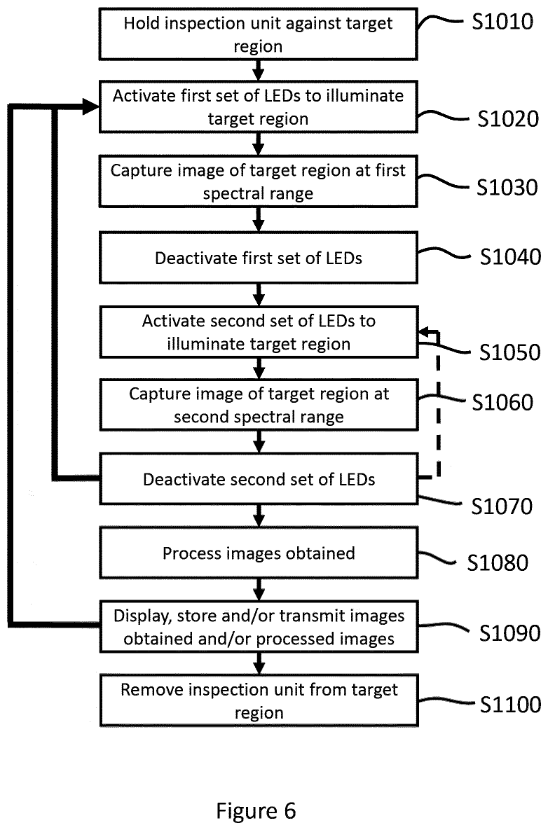

FIG. 6 is a flow chart of a method of imaging a target region using the imaging apparatus shown FIG. 1;

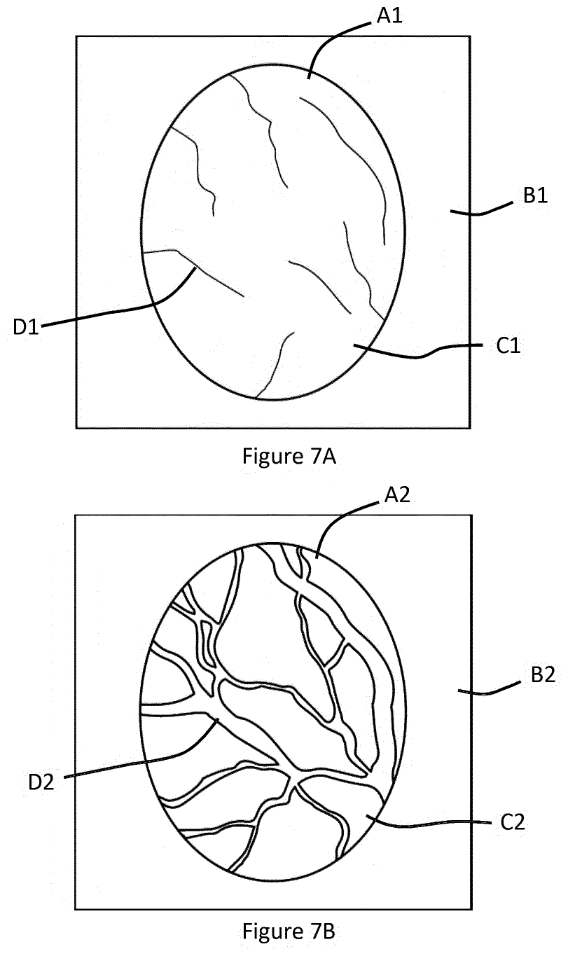

FIG. 7A is a representation of a first example image captured using the imaging apparatus shown in FIG. 1;

FIG. 7B is a representation of a second example image captured using the imaging apparatus shown in FIG. 1;

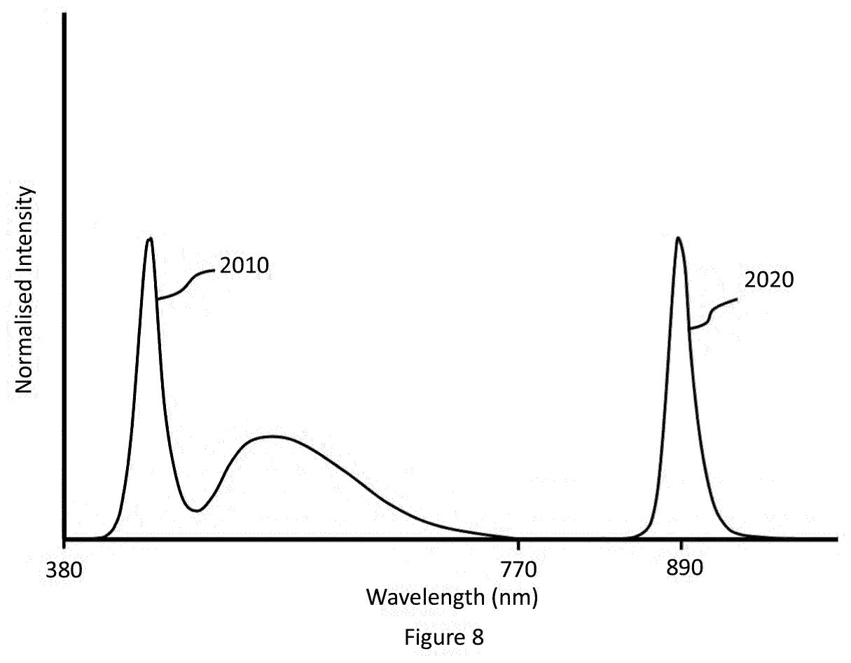

FIG. 8 shows spectral distributions for a further embodiment of a sensor module; and

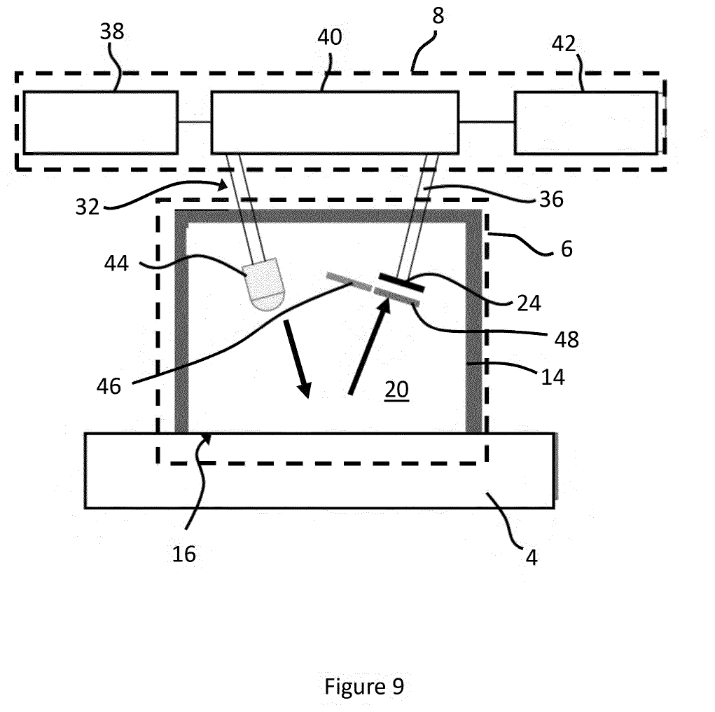

FIG. 9 is a schematic representation of key components of a further embodiment of imaging apparatus.

FIG. 1 shows apparatus 2 for imaging blood perfusion at a target region of skin tissue being used to image blood perfusion at a region of a patient's wrist 4. Other areas of a persons or animals body and other types of tissue such as wound tissue could, of course, be investigated. The apparatus comprises an inspection unit 6 and a display unit 8. The display unit 8 in the embodiment shown comprises a portable device, in particular, a tablet device, having an integrated screen 10. The inspection unit 6 and the display unit 8 are connected by a lead 12. A wireless connection could of course be utilised.

FIG. 2 shows schematic representation of the inspection unit 6. The inspection unit 6 comprises an oblong housing 14. The housing 14 has an opening at one end, which defines a viewing aperture 16 of the inspection unit 6, and a sensor module 18 at the other end. The housing 14 comprises four side walls 14a, 14b, 14c, 14d and an end wall 14e to which the sensor module 18 is secured such that the sensor module 18 is located within the space 20 defined by the walls 14a, 14b, 14c, 14d, 14e. The walls 14a, 14b, 14c, 14d, 14e of the housing 14 are opaque and are substantially not reflective. In the embodiment shown, the walls 14a, 14b, 14c, 14d, 14e are formed from rigid black plastic. The housing 14 provides structural support for the sensor module 18 when the inspection unit 6 is held against the patient's wrist while shielding the area enclosed by the housing 14 from ambient light. The housing 14, or the portion of the housing 14 at which the aperture 16 is provided, may be disposable so that it may be replaced after use and/or between use on different patients or when used repeatedly on the same patient.

The sensor module 18 is shown in FIG. 3. The sensor module 18 comprises a light source 22 and an imaging device comprising a camera module 24.

The light source 22 comprises two sets of light emitting diodes (LEDs) 26, 28 which are mounted to the front of the camera module 24 such that they emit light at least in a direction which is away from the camera module 22 towards the aperture 16. The first set of LEDs 26 comprises eight LEDs which are arranged about the circumference of a lens 30 of the camera module 24. In the embodiment shown, the LEDs of the first set of LEDs 26 are arranged in diametrically opposed pairs. The second set of LEDs 28 also comprises eight LEDs which are also arranged about the circumference of the lens 30. The second set of LEDs 28 are spaced radially further outwardly from the lens 30 than the first set of LEDs 26 and are also arranged in diametrically opposed pairs. The first and second sets of LEDs 26, 28 are arranged to provide reasonably uniform illumination of a target region of issue placed at the aperture 16 from within the housing 14. The sets of LEDs 26, 28 are connected by power supply wires 32, which comprise part of the lead 12, to the display unit 8.

A shield 34 is located between the sets of LEDs 26, 28 and the camera module 24 to inhibit light emitted by the LEDs from being transmitted to the camera module 24 directly (i.e. prior to being absorbed or reflected) and helps shield the camera module 24 from any ambient light which may enter the housing 14. In the present embodiment, both sets of LEDs 26, 28 have an angular emission range of between +/−45 degrees and so emit light in a direction away from the camera module 24 towards the aperture 16. In addition, both sets of LEDs 26, 28 are located in front of the camera module 24. Consequently, very little light is transmitted from the LEDs 26, 28 directly towards the camera module 24. Therefore, in other embodiments, a shield may not be required. The optics of the camera module 24 may also be configured to reduce the impact of directly transmitted light and/or other light which would have an adverse impact on images captured by the camera module 24. The camera module 24 is connected by a flex cable 36, which comprises part of the lead 12, to the display unit 8. In the embodiment shown, the camera module 24 has a fixed focal length and is arranged within the housing such that when the housing 14 is held against the target region of tissue, the cameras module 24 is spaced from the target region at a distance which corresponds to the focal length. The target region of tissue is therefore in focus.

Each LED of the first set of LEDs 26 is configured to emit light having a predefined first spectral range. In the embodiment shown, the first spectral range of the light emitted by the first set of LEDs is light having at distribution of wavelengths between 600 nm and 750 nm. The range is within the visible red spectrum. The normalized intensity profile 1010 of the light emitted by the first set of LEDs 26 over the spectral range is shown in FIG. 4 (not shown to scale). Light is emitted by each of the LEDs of the first set of LEDs 26 over the entire first spectral range. That is to say, each of the LEDs of the first set of LEDs emits light having a distribution of wavelengths between 600 nm and 750 nm in accordance with the intensity profile shown in FIG. 4. The normalized intensity profile has a peak wavelength at 660 nm and the intensity drops from the peak to substantially zero at each of the limits, 600 nm and 750 nm respectively, of the spectral range.

Each LED of the second set of LEDs 28 is configured to emit light having a predefined second spectral range. In the embodiment shown, the second spectral range of the light emitted by the first set of LEDs is light having at distribution of wavelengths between 850 nm and 1000 nm. The range is within the near-infrared spectrum. The normalized intensity profile 1020 of the light emitted by the second set of LEDs 28 is shown in FIG. 4 (not to scale). The normalized intensity profile has a peak wavelength at 890 nm.

As referred to above, FIG. 4 shows a normalized intensity distribution profile 1010 of light emitted by the first set of LEDs 26 and a normalized distribution profile 1020 of light emitted by the second set of LEDs 28. The distribution profiles 1010, 1020 are not to scale, but demonstrate that the spectral ranges of light emitted by each of the two sets of LEDs 26, 28 do not overlap.

FIG. 5 is a schematic representation of some of the components of the apparatus 4. Components of the inspection unit 6 and the display unit 8 are enclosed, respectively, by broken lines.

The display unit 8 further comprises a power source 38, such as a battery, mains connection or the like, a controller or controlling element in the form of a processor 40 configured to drive each set of LEDs 26, 28 independently (i.e. each set of LEDs 26, 28 may be activated independently of the other set of LEDs 26, 28) and to process an output of the camera module 24, and an output device 42 which is configured to display an output from the processor 40 on the screen 10 of the display unit 8.

As illustrated in FIG. 5, the sets of LEDs 26, 28 and the camera module 24 are arranged such that at least some of the light emitted by the LEDs travels within the housing 14 in the direction of the aperture 16 and at least some of the light reflected by skin tissue of the wrist at the aperture 16 travels back within the housing 16 to be received by the camera module 24. Light received by the camera module 24 passes through the lens 30 (shown in FIG. 3) before being received by an imaging sensor (not shown) within the camera module. The imaging sensor is configured to detect light emitted by LEDs from both the first set of LEDs 26 and the second set of LEDs 28. In the embodiment shown, the camera module 24 comprises an imaging sensor in the form of a charge coupled device (CCD). Other sensors such as a complementary metal-oxide semiconductor (CMOS) or the like configured to detect light across a broad spectral range could of course be utilised. For example, the imaging sensor may be a sensor configured to detect light over a spectral range of wavelengths between 380 nm and 1350 nm.

A flow chart illustrating a method of imaging blood within a target region of skin tissue is shown in FIG. 6.

In use, at step S1010, the inspection unit 6 is held in contact with a target region of skin tissue which is to be imaged such that the skin tissue occludes the aperture 16, as shown in FIG. 1. The target region of skin tissue therefore seals against the edges of the housing 14 defining the aperture 16 so that the amount of ambient light which enters the housing 16 through the aperture 16 is restricted or ambient light is prevented from entering the housing altogether.

At step S1020, the first set of LEDs 26 is activated to illuminate the target region of skin tissue at the aperture 16 with light having the spectral distribution 1010 shown in FIG. 4. At least some of the light emitted by the first set of LEDs 20 is transmitted directly to the skin tissue. The remaining light is transmitted to the walls 14a, 14b, 14c, 14d, 14e of the housing 14, which are black and substantially not reflective (i.e. highly absorbent) and so absorb substantially all or all of the light which is not transmitted directly to the skin tissue. Light which reaches the skin tissue is either absorbed or reflected by the skin tissue. At least some of the light reflected by the skin tissue is reflected towards the camera module 24. The housing 14 therefore defines a light path that extends from the light source 22 to the aperture 16 and from the aperture 16 to the camera module 24. The housing 14 is opaque to the wavelengths of light at which analysis occurs and so provides a shroud that prevents all or substantially all ambient light from entering the housing 14 and reaching the camera module 24. Any light received at the camera module 24 originated from the first set of LEDs 26 and so has a wavelength within the spectral range 1010 shown in FIG. 4. Although ingress of some ambient light can be tolerated, the amount should be small compared with the intensity of the light emitted by the LEDs 26. The ratio of light emitted by the first set of LEDs 26 received at the cameral module to ambient light received at the camera module should be not less than 5:1, for example not less than 10:1. In other embodiments, any adverse effect of ambient light entering the housing 14 could be mitigated by obtaining a preliminary image of the target region without any illumination from by either the sets of LEDs 26, 28. The preliminary image can be used to calibrate the subsequent images obtained for analysis of the target region or used to inform a user that the housing should be repositioned to reduce the amount of ambient light entering the housing 14.

At step S1030, light reflected by the skin tissue which passes through the lens 30 of the camera module 24 is received by the image sensor and processed by the processor 40. In the present embodiment, the camera module is configured to have an ISO setting of 100 and a shutter speed of 72 ms when the first set of LEDs 26 is used to illuminate the target region of tissue. Other imaging settings could of course be utilised in order to obtain an image of the target region of tissue. The first set of LEDs 26 are activated using a drive current of 1 mA during image capture. The image is then sent by the output device 42 to the imaging unit 8 for storage and/or display on the screen 10. The first set of LEDs 26 is then deactivated at step S1040.

At step S1050, the second set of LEDs 28 is activated to illuminate the target region of skin tissue at the aperture 16 with light having the spectral range 1020 shown in FIG. 4. At least some of the light reflected by the skin tissue is reflected towards the camera module 24. The remaining light is absorbed by the walls 14a, 14b, 14c, 14d, 14e of the housing 14, or the skin tissue, as described above.

At step S1060, light reflected by the skin tissue which passes through the lens 30 of the camera module 24 is received by the image sensor and processed by the processor 40. In the present embodiment, the camera module is configured to have an ISO setting of 100 and a shutter speed of 48 ms when the second set of LEDs 28 is used to illuminate the target region. Other imaging settings could of course be utilised in order to obtain an image of the target region of tissue. The second set of LEDs 26 are activated using a drive current of 1 mA during image capture. The image is then sent by the output device 42 to the imaging unit 8 for storage and/or display on the screen 10. The second set of LEDs 28 is then deactivated, at step S1070.

Once an image at each spectral range has been captured, steps S1020 through S1070 can be repeated to capture multiple images at each spectral range, if desired.

Once one or more images at each spectral ranged has been captured, the images may be optionally processed at step 1080, for example, to produce a combined image. Successive images taken at each spectral range may be paired. At step S1090, the images are subsequently displayed, stored and/or transmitted individually, or as paired images (for example, displayed side-by-side) or as a combined image.

At step S1100, the inspection unit 6 is removed from the target region of skin tissue once inspection is complete.

FIG. 7A and FIG. 7B are illustrative diagrams of images captured using light emitted by the first and second sets of LEDs 26, 28.

FIG. 7A illustrates a first image captured by the camera module 24 using light emitted by the first set of LEDs 26 having the normalized intensity distribution profile 1010 shown in FIG. 4. The image has a central oval portion A1 which corresponds to the portion of the target region of skin tissue which is illuminated by the first set of LEDs 26. The image has a peripheral portion B1 corresponds to a less-well illuminated region. The boundary between the two regions may be well defined, as shown in FIG. 7A, graduated or even non-existent if the whole area is sufficiently well illuminated.

Within the central portion of the image, there are lighter regions C1 and darker regions D1 (which appear as isolated lines on the image). The amount of light received by the camera module 24 is inversely proportional to the amount of light absorbed by the skin tissue. Therefore, darker regions of an image indicate areas of high absorbance (i.e. low reflectivity) whereas lighter regions of the image indicate areas of low absorbance (i.e. high reflectivity). The image, which is captured over the red spectral range of visible, therefore shows features such as skin defects, scarring, moles and underlying blood vessels carrying deoxygenated haemoglobin as dark regions on the image. Although other blood vessels are discernible as lines D1 on the image, their appearance is very faint and it is extremely difficult, or impossible, to distinguish between vessels and other features clearly.

FIG. 7B illustrates a second image captured by the camera module 24 using light emitted by the second set of LEDs 28 having the normalized distribution profile 1020 shown in FIG. 4. The image has a central portion A2 and an outer portion B2 similar to the first image. Within the central portion A2 there are light regions C2 bounded by darker regions D2 (which, although not darker in the representation, are the narrow reticulated “pathways” in the second image). The darker regions D2 in the second image are blood vessels within the skin tissue carrying oxygenated blood. In the embodiment shown, the LEDs comprising the second set of LEDs 28 emit light having a spectral range which corresponds with a peak in the absorption spectra of oxygenated haemoglobin. The second image therefore provides a clear indication of the quantity and location of oxygenated blood vessels. Furthermore, since the amount of light absorbed is proportional to the amount of oxygenated blood within the vessels, the second image provides a reliable indication of the amount of oxygenated blood within the vessels.

Features having prominence in the image shown in FIG. 7A are unlikely to be blood vessels carrying oxygenated blood. They may, for example, be features such as skin defects, moles, tissue or de-oxygenated blood, for example, that absorb light equally at the first spectral range and the second spectral range, for example. Such features can therefore be disregarded either by a clinician or automatically by processing the images obtained using image recognition software.

Images obtained at either or both of the first and second spectral ranges can be processed to enhance features of interest. For example, the brightness of an image can be adjusted or the contrast enhanced. An image may also be adjusted to enhance or mute particular colours. Images may also be filtered for noise. Image recognition techniques may also be utilised to identify certain features of interest.

In a further embodiment, the LEDs of the first set of LEDs 26 may comprise LEDs that emit white light having a relatively wide spectral range between 380 nm and 770 nm. Illumination using white light produces a high-quality image that is visible to the naked eye and may utilise the full sensing range of typical CCD imaging sensors thereby maximising the amount of light processed to produce an image. FIG. 8 shows an example of the spectral ranges of white light and infrared light.

In order to identify blood vessels, the LEDs of the second set of LEDs 28 may comprise LEDs that emit light having a spectral range of wavelengths in region that is absorbed or reflected by blood. For example, LEDs which emit light in the green spectral range of between 495 nm and 570 nm, and/or the red spectral range of between 600 nm and 750 nm, and or the near-infrared spectral range of between 850 nm and 1000 nm. Furthermore, LEDs may be used that emit light having a spectral range within the near-infrared window of biological tissue of between 650 nm and 1350 nm. Such light is absorbed poorly by tissue surrounding blood vessels and so will provide good tissue penetration, but will be absorbed well by blood within the tissue. The LEDs of the second set of LEDs 28 may have a peak wavelength of 890 nm, as described with respect to the embodiment shown or 910 nm or 950 nm may be used. In further embodiments, a third set of LEDs may be provided for illuminating the target region with light having a third spectral range that is different from the spectral range of the first and second sets of LEDs 26, 28 and for capturing an image or images at the third spectral range. The third spectral range may be associated with high absorption by other chemical species within tissue and so can be used to identify the presence of other chemical species within tissue. Steps S1050 to S1070 can be repeated using the third set of LEDs.

FIG. 9 is a schematic representation of a variation of the embodiment described above in which the two sets of LEDs are replaced with a single set of LEDs 44 which emits light over a single broad spectral range which encompasses both visible and infrared light. First and second filters 46, 48 are arranged within the housing 14 such that they can be placed sequentially in front of the camera module 24. For example, the first filter 46 may restrict light passing through the filter to light within the red visible spectral band having a spectral range between 600 nm and 750 nm (i.e. corresponding to the spectral range emitted by the first set of LEDs described in connection with the embodiment described above). The second filter 48 may restrict light passing through the filter to light within the infrared region having a spectral range between 850 nm and 1000 nm (i.e. corresponding to the spectral range emitted by the second set of LEDs described in connection with the embodiment described above).

The processor 40 is configured to drive the single set of LEDs 44 and to automatically move the filters sequentially in front of the camera module 24 when capturing images. Alternatively, the positioning of the filters may be controlled manually.

In use, sequential images are captured with each of the filters 46, 48 disposed in front of the camera module 24 respectively. The images are then processed as described above.

In the embodiments described above, LEDs are used for illumination. However, it will be appreciated that a wide range of light sources could be used such as incandescent light sources, discharge lamps, fluorescent lamps, solid state light sources, organic LEDs, polymer LEDs, laser diodes or super-luminescent diodes. Surface-mount LEDs may be suitable because of they are compact, widely available, inexpensive, reliable, easily integrated and have a low power consumption. Although the described embodiments describe sets of LEDs comprising multiple LEDs, a set of LEDs could comprise a single LED. A set of LEDs may be comprise a chip on which multiple LEDs having different spectral ranges are mounted. Such devices are compact and cost-effective. In the embodiments described above, the first and second spectral ranges do not overlap. In other embodiments, there may be an overlap of the first and second spectral ranges provided that there are sufficient differences in the absorption of light by tissue across each of the spectral ranges so that images produced at each of the spectral ranges are sufficiently different to distinguish features within the images.

A diffuser or lens arrangement configured to diffuse light emitted by the first and or second LEDs may be provided in order to provide a uniform illumination of the target region of tissue.

The housing may be made from a variety of suitable materials including non-transparent materials or other materials that are painted or covered by a non-transparent material. For example, the housing may comprise plastic, metal, cardboard or paper. In the embodiments described, the housing is oblong having a single aperture at one end. Other shapes could of course be utilised. Likewise the shape and number of apertures could be different.

The embodiments described above comprise a power source in the form or a battery, mains connection or the like. Alternative or additional power sources may be used such as one or more capacitors, fuel cells or energy generators, which generate energy, for example, from the movement of the wearer, e.g. based on some piezo elements or the like, from temperature differences and heat generated by the user or the environment, using, for example, thermopiles, or from light, using, for example, photovoltaic cells, or other energy generating systems, for example clockwork type mechanisms which can be charged by the user. Any battery used may be non-rechargeable or rechargeable. Recharging may be conducted using wired or contactless charging techniques.

The embodiments described above comprise an inspection unit that is movable with respect to the display unit. The display unit is a portable device having an integrated touch-screen which forms a control interface with a user for selecting analysis functions and displaying captured/processed images and data. In other embodiments, the display unit may have dedicated buttons for selecting analysis functions. In further embodiments, the inspection unit and display unit may be incorporated into a portable hand-held device having a single housing in which the components of the inspection unit and the display unit are housed.

It will be appreciated that throughout this specification reference is made to a wound. It is to be understood that the term wound is to be broadly construed and encompasses open and closed wounds in which skin is torn, cut or punctured or where trauma causes a contusion, or any other superficial or other conditions or imperfections on the skin of a patient or otherwise that benefit from reduced pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

In the drawings like reference numerals refer to like parts.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any foregoing embodiments. The disclosure extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference in their entireties.

1. Imaging apparatus for imaging blood vessels within a target region of tissue, comprising:
a housing having an aperture which, in use, is placed against a target region of tissue such that the target region of tissue occludes the aperture;
a light source arranged to illuminate at least a portion of a target region of tissue occluding the aperture, the light source is configured to provide illuminating light having a predetermined first spectral range and to provide illuminating light having a predetermined second spectral range which is different from the first spectral range;
an imaging device arranged to receive illuminating light reflected by the target region of tissue occluding the aperture, wherein the imaging device is configured to generate an image output at the first spectral range and an image output at the second spectral range; and
a controlling element arranged to selectively control the imaging device and/or the light source to sequentially capture at least one image generated by the imaging device at the first spectral range and at least one image generated by the imaging device at the second spectral range.

2. The imaging apparatus of claim 1, wherein the first spectral range and the second spectral range do not overlap.

3. The imaging apparatus of claim 1 or 2, wherein the imaging device is configured to detect light across a continuous spectral range which encompasses the first spectral range and the second spectral range.

4. The imaging apparatus of any one of the preceding claims, wherein the controlling element is configured to control the light source to provide illuminating light at only the first spectral range during capture of the at least one image at the first spectral range and to provide illuminating light at only the second spectral range during capture of the at least one image at the second spectral range.

5. The imaging apparatus of any one of the preceding claims, wherein the first spectral range corresponds to a spectral range associated with visible light.

6. The imaging apparatus of claim 5, wherein the first spectral range corresponds to a spectral range associated with visible red light.

7. The imaging apparatus of any one of the preceding claims, wherein the second spectral range corresponds to a spectral range encompassing a wavelength absorbed by blood.

8. The imaging apparatus of any one of the preceding claims, wherein the second spectral range corresponds to a spectral range associated with infrared light.

9. The imaging apparatus of claim 8, wherein the second spectral range corresponds to light having a wavelength between 850 nm and 1000 nm.

10. The imaging apparatus of any one of the preceding claims, wherein the imaging device comprises a charge coupled device and/or a complementary metal-oxide semiconductor.

11. The imaging apparatus of any one of the preceding claims, wherein the light source comprises at least a first light emitter configured to emit light having the first spectral range and a second light emitter configured to emit light having the second spectral range.

12. The imaging apparatus of claim 11, wherein the controlling element is configured to selectively activate the first light emitter during capture of the image at the first spectral range and to selectively activate the second light emitter during capture of the image at the second spectral range.

13. The imaging apparatus of any one of claims 1 to 10, wherein the light source is configured to emit light having a spectral range which encompasses the first spectral range and the second spectral range.

14. The imaging apparatus of claim 13, wherein the imaging device comprises at least a first filter arranged to transmit light within the first spectral range and a second filter arrange to transmit light within the second spectral range, wherein the controlling element is configured to selectively apply the first filter during capture of the image at the first spectral range and to apply the second filter during capture of the image at the second spectral range.

15. The imaging apparatus of any one of the preceding claims, wherein the housing is a rigid housing and the imaging device is secured to the housing such that, when the aperture is placed against the target region of tissue, the imaging device is spaced from the target region of tissue by a predetermined distance.

16. The imaging apparatus of claim 15, wherein the imaging device comprises a lens having a predefined focal length and the predetermined distance is such that the lens is spaced from the target region of tissue by a distance that is equal to the focal length.

17. The imaging apparatus of any one of the preceding claims, wherein the housing is opaque to light having a wavelength which is within the first spectral range and is opaque to light having a wavelength which is within the second spectral range.

18. The imaging apparatus of any one of the preceding claims, wherein the housing defines a light path extending from the light source to the aperture and from the aperture to the imaging device and the housing is arranged to shield the light path from ambient light.

19. The imaging apparatus of any one of the preceding claims, wherein the light source and/or the imaging device are disposed within the housing.

20. The imaging apparatus of any one of the preceding claims, wherein the apparatus is a hand-held device comprising the light source, the imaging device, the housing and the integrated screen.

21. Imaging apparatus for imaging blood vessels within a target region of tissue, comprising:
a housing having an aperture which, in use, is placed against a target region of tissue such that the target region of tissue occludes the aperture;
a light source arranged to illuminate at least a portion of a target region of tissue occluding the aperture, the light source is configured to provide illuminating light having a predetermined first spectral range and to provide illuminating light having a predetermined second spectral range which is different from the first spectral range;
an imaging device arranged to receive illuminating light reflected by the target region of tissue occluding the aperture, wherein the imaging device is configured to generate an image output at the first spectral range and an image output at the second spectral range; and
a controlling element arranged to selectively control the imaging device and/or the light source to sequentially capture at least one image generated by the imaging device at the first spectral range and at least one image generated by the imaging device at the second spectral range,
the controlling element further arranged to combine the at least one image captured at the first spectral range and the at least one image captured at the second spectral range to produce a composite image in which blood vessels within the target region of skin tissue can be distinguished.

22. A method of imaging blood vessels within a target region of tissue using an imaging apparatus comprising a housing having an aperture, comprising the steps:
holding the housing against a target region of tissue such that the target region of tissue occludes the aperture;
illuminating the target region of tissue occluding the aperture using light having at least a predetermined first spectral range;
capturing at least one image of the target region of tissue at the first spectral range;
illuminating the target region of tissue using light having at least a predetermined second spectral range which is different from the first spectral range; and
capturing at least one image of the target region of tissue at the second spectral range.

23. The method of claim 22, wherein the light having at least the predetermined first spectral range is light having only the first spectral range.

24. The method of claim 22 or 23, wherein the light having at least the predetermined second spectral range is light having only the second spectral range.

25. The method of claim 22, wherein the step of capturing at least one image of the target region of tissue at the first spectral range comprises the step of filtering the light having at least the first spectral range such that only light having the first spectral range is transmitted for image capture.

26. The method of claim 22 or 25, wherein the step of capturing at least one image of the target region of tissue at the second spectral range comprises the step of filtering the light having at least the second spectral range such that only light having the second spectral range is transmitted for image capture.

27. The method of any one of claims 22 to 26, wherein the first spectral range and the second spectral range do not overlap.

28. The method of any one of claims 22 to 27, wherein the first spectral range corresponds to a spectral range associated with visible light.

29. The method of claim 28, wherein the first spectral range corresponds to a spectral range associated with visible red light.

30. The method of any one of claims 22 to 29, wherein the second spectral range corresponds to a spectral range encompassing a wavelength absorbed by blood.

31. The method of any one of claims 22 to 30, wherein the second spectral range corresponds to a spectral range associated with infrared light.

32. The method of claim 31, wherein the second spectral range corresponds to a light having a wavelength between 850 nm and 1000 nm.

33. The method of any one of claims 22 to 32, further comprising the step of automatically combining the at least one image captured at the first spectral range and the at least one image captured at the second spectral range to produce a composite image in which blood vessels within the target region of skin tissue can be distinguished.

34. A method of imaging blood vessels within a target region of tissue using an imaging apparatus comprising a housing having an aperture, comprising the steps:
illuminating a target region of tissue occluding the aperture of the housing held against the target region of tissue using light having at least a predetermined first spectral range;
capturing at least one image of the target region of tissue at the first spectral range;
illuminating the target region of tissue using light having at least a predetermined second spectral range which is different from the first spectral range; and
capturing at least one image of the target region of tissue at the second spectral range.

35. The method of claim 34, wherein the light having at least the predetermined first spectral range is light having only the first spectral range.

36. The method of claim 34 or 35, wherein the light having at least the predetermined second spectral range is light having only the second spectral range.

37. The method of claim 34, wherein the step of capturing at least one image of the target region of tissue at the first spectral range comprises the step of filtering the light having at least the first spectral range such that only light having the first spectral range is transmitted for image capture.

38. The method of claim 34 or 37, wherein the step of capturing at least one image of the target region of tissue at the second spectral range comprises the step of filtering the light having at least the second spectral range such that only light having the second spectral range is transmitted for image capture.

39. The method of any one of claims 34 to 38, wherein the first spectral range and the second spectral range do not overlap.

40. The method of any one of claims 34 to 39, wherein the first spectral range corresponds to a spectral range associated with visible light.

41. The method of claim 40, wherein the first spectral range corresponds to a spectral range associated with visible red light.

42. The method of any one of claims 34 to 41, wherein the second spectral range corresponds to a spectral range encompassing a wavelength absorbed by blood.

43. The method of any one of claims 34 to 42, wherein the second spectral range corresponds to a spectral range associated with infrared light.

44. The method of claim 43, wherein the second spectral range corresponds to a light having a wavelength between 850 nm and 1000 nm.

45. The method of any one of claims 34 to 44, further comprising the step of automatically combining the at least one image captured at the first spectral range and the at least one image captured at the second spectral range to produce a composite image in which blood vessels within the target region of skin tissue can be distinguished.

46. A method of imaging blood vessels within a target region of tissue using an imaging apparatus comprising a housing having an aperture, comprising the steps:
illuminating a target region of tissue occluding the aperture of the housing held against the target region of tissue using light having at least a predetermined first spectral range;
capturing at least one image of the target region of tissue at the first spectral range;
illuminating the target region of tissue using light having at least a predetermined second spectral range which is different from the first spectral range;
capturing at least one image of the target region of tissue at the second spectral range; and
automatically combining the at least one image captured at the first spectral range and the at least one image captured at the second spectral range to produce a composite image in which blood vessels within the target region of skin tissue can be distinguished.
